(19) United States
(12) Patent Application Publication
(10) Pub. No.: US 2007/0112420 A1
(43) Pub. Date: May 17, 2007

(54) Detachable therapeutic tube

(75) Inventor: Robert C. LaDuca, Santa Cruz, CA (US)

Correspondence Address: LEVINE BAGADE HAN LLP, 2483 EAST BAYSHORE ROAD, SUITE 100, PALO ALTO, CA, 94303, US

(73) Assignee: Duke Fiduciary LLC, Santa Cruz, CA (US)

(21) Appl. No.: 11/274,623

(22) Filed: Nov. 14, 2005

(51) Int. Cl. A61F 2/06

(52) U.S. Cl. 623/1.44

(57) ABSTRACT

The invention provides a detachable tube deliverable to a body lumen using a catheter. The detachable tube is detached from the catheter using an expandable stent disposed within the tube to break perforations or attachments engineered to connect the tube to the catheter until the stent is expanded. The detached tube and interdisposed stent reside against the lumen surface, the stent holding the tube against the lumen surface, and the stent itself in contact with the interior wall of the tube. The tube can be constructed from extracellular matrix materials or other polymeric therapeutic biodegradable and/or drug eluting materials. Treatment of a lumen surface having a defect is accomplished by delivering the therapeutic tube to the site of defect in the lumen, delivering an expandable stent to within the tube, expanding the stent to break the tube attachments to the delivery catheter, and removing any catheter, guidewires or dilators to leave an expanded stent disposed against the interior wall of the tube, itself being in contact with the luminal surface. Once in place, the tube can impart drug and other bioactive agents to the lumen surface to provide an opportunity for the lumen to heal and otherwise regenerate healthy luminal tissue.

### CROSS-REFERENCES TO RELATED APPLICATIONS

This application does not draw priority from any earlier applications.

### BACKGROUND OF THE INVENTION

In 2003 and 2004 the Food and Drug Administration approved the two different drug-eluting stents for angioplasty procedures to open clogged coronary arteries. A drug-eluting stent is a metal stent that has been coated with a pharmacologic agent that interferes with restenosis, or the reblocking of the artery. Each year close to 1 million angioplasty procedures are performed, and of those some 30% of patients experience restenosis within one year, requiring further treatment such as repeat angioplasty or surgery. With the advent of drug eluting stents that elute anti-restenotic drugs, the incidence of restenosis after stent placement has been reduced to single digits.

Effectiveness of the drug-eluting stent depends at least in part on the type of metal stent used, the coating selected and the pharmacological agent selected, how the agent is released at the site, and whether the stent has been properly placed in the artery to prevent the complications of blood clots or sub-acute thrombosis. Early trials using drug-eluting stents indicate that they are much more successful at treating patients than bare stents alone. Currently available stents include a paclitaxel-eluting stent (that releases the chemotherapeutic drug paclitaxel) and a sirolimus-eluting stent (that releases the immunosuppressant simolimus). Both stents are bare metal stents that have been coated with a slow to moderate release drug formulation embedded in a polymer. The drug is selected based on its ability to slow or inhibit the process of restenosis, which is sometimes characterized as epithelial cell hyperplasia in response to the injury of angioplasty or stent placement. Both products have proven successful in clinical trials in comparison with bare metal stents or angioplasty alone. Presently, data from clinical trials indicates a four-fold reduction in the incidence of restenosis with medicated stents.

One side effect of drug-eluting stents is that because the drugs currently used in the drug-eluting stents delay endothelisation by inhibiting fibroblast proliferation, they put the patient at risk for stent thrombosis within the 6 months following the stent placement. For this reason patients implanted with drug-eluting stents receive anti-coagulants such as clopidogrel or ticlopidine for up to 6 months following placement of the device, to prevent thrombosis which is the blood from reacting to the new device by thickening and clogging up the newly expanded artery. If the system works, a smooth thin layer of endothelial cells (which is the inner lining of the blood vessel) grows over the stent during this period and the device is incorporated into the artery, reducing the tendency for clotting.

It would be advantageous to develop other ways to conduct angioplasty procedures in which devices and drug administration were optimized to effect maximum beneficial outcome for treated patients.

### BRIEF DESCRIPTION OF THE DRAWINGS

FIG. 1 depicts the basic tube delivery catheter.

FIG. 2 depicts a tube delivery catheter with a dilator therein disposed.

FIG. 3 depicts a tube delivery catheter having a nose cone tip, positioned over a guidewire, and also having a stent delivery catheter with a partly expanded stent covered balloon.

FIG. 4 depicts a tube delivery catheter having a stent delivery catheter disposed within it, with a stent covered balloon disposed within the detachable tube.

FIG. 5 depicts a tube delivery catheter being detached from the delivered tube, the delivered tube having a fully expanded stent within the tube and contacting the interior wall of the expandable detached tube.

### SUMMARY OF THE INVENTION

The invention provides a device comprising a tube comprising extracellular matrix, the tube having an interior wall, an exterior wall, a distal end, and a proximal end, wherein the tube is detachable from a catheter at the proximal end, and can be placed in a body lumen.

The tube comprises perforations or other detachable attachments at the proximal end for detachment from the catheter and can also comprise these detachable attachments at the distal end of the tube where the tube resides proximal to the distal tip of the catheter, for example, with a dilator or nose cone configuration of catheter. The tube is detachable from the catheter by breaking the perforations or detachable attachments. The tube is expandable upon pressure from the interior wall of the tube that breaks the perforations or other attachments and the tube detaches from the catheter. Application of pressure on the interior wall of the tube can comprise expansion of an expandable stent disposed within the tube, with the stent contacting the interior wall of the tube. The stent can expand within the tube until the exterior wall of the tube contacts a surface of the body lumen. The tube material can be mammalian or synthetic extracellular matrix. In general any expandable therapeutic polymeric material capable of biodegrading in the body can be used for the tube material. Accordingly, the tube material can further comprise an extruded material, a biodegradable material, and a drug eluting material.

The invention provides a catheter device having a distal end comprising a detachable portion, the detachable portion comprising a tube placeable in the lumen of a body, the tube having an interior wall and exterior wall, and a distal end and a proximal end detachable from the catheter, the tube comprising an expandable therapeutic polymeric material. The material can be selected from the group consisting of extracellular matrix derived from a mammal, synthetic extracellular matrix, an extruded material, a biodegradable material, and a drug eluting material.

The invention also provides a method of repairing a defect in a lumen surface comprising placing an expandable tube having an interior wall and an exterior wall and an expandable stent disposed within the tube at the site of defect, and expanding the expandable stent until the exterior wall of the tube is in contact with the lumen surface and the stent is in contact with the interior wall of the tube. The tube comprises a therapeutic polymeric material. The material can be selected from the group consisting of extracellular matrix derived from a mammal, synthetic extracellular matrix, an extruded material, a biodegradable material, and a drug eluting material. The stent comprises a metal or metal alloy and is expandable with an inflatable balloon or is self-expanding.

The invention further provides a method of repairing a defect in a lumen surface comprising placing a guidewire in the lumen proximal to the defect, sliding over the guidewire a first catheter comprising an expandable tube, the tube comprising perforations for detachment from the catheter as an intact tube, the tube having an interior wall and an exterior wall, inserting an expandable stent within the expandable tube using a second catheter disposed within the first catheter, the second catheter also sliding over the guidewire, and expanding the stent within the tube until the exterior wall of the tube is in contact with the lumen surface and the stent is in contact with the interior wall of the tube. During the expanding step, the stent expands within the tube and the tube detaches from the first catheter. The tube comprises a therapeutic polymeric material. The material can be selected from the group consisting of extracellular matrix derived from a mammal, synthetic extracellular matrix, an extruded material, a biodegradable material, and a drug eluting material. The stent is self expanding or is expanded by an expansion means. The stent can comprise a metal or metal alloy, for example nitinol.

The invention provides a method of making a device for delivery to a body lumen, the device comprising a tube comprising an expandable therapeutic polymeric material, the tube having an interior wall and an exterior wall, and proximal and distal ends, comprising forming a tube of the expandable therapeutic polymeric material, applying perforations or attachments in the tube at the proximal end to make the tube attached but detachable from a delivery catheter, and attaching the perforated tube to a catheter for delivery to the body lumen. The method further comprises applying perforations in the tube at the distal end. The step of forming comprises a process selected from the group consisting of extruding, sewing, laminating, pressing, freeze-drying, gluing, and molding. The expandable therapeutic polymeric material can be selected from the group consisting of extracellular matrix derived from a mammal, synthetic extracellular matrix, an extruded material, a biodegradable material, and a drug eluting material.

The invention further provides a method of drug delivery comprising placing a tube in the lumen of a living body, wherein the tube comprises a therapeutic polymeric material having at least one bioactive agent or drug capable of release in the lumen of the body.

### DETAILED DESCRIPTION OF THE INVENTION

The invention contemplates a casing or tube-like structure at the distal portion (tip or end) of a catheter shaft. The tube can be detachably attached at a proximal point of the tube to the catheter shaft. If the tube is located at the distal portion of the catheter but not at the very end of it, the tube may be detachably attached to the catheter at both a proximal and distal point of the tube.

Included with the tube, and in consideration of its placement within the catheter shaft is a means for detaching the tube from the catheter shaft. For example, perforations in the material or loose attachments to the material can provide the opportunity to tear or break the tube from the main body of the catheter. During the stenting procedure, a stent is disposed within the tube and the stent is expanded within the tube so that it contacts the interior walls of the tube. As the tube is expanded, the tube separates from the catheter body, for example by breaks or tears in perforations or string attachments that cause it to detach from the catheter material. The stent is then further expanded so that eventually the exterior walls of the tube contact the wall of the lumen under repair. The stent can be expanded by any means possible to effect stent expansion. A few common examples would include a stent disposed over an inflatable balloon so that the stent expands as the balloon expands, or the stent can be a self-expanding stent having spring-like properties.

Method and apparatus for releasing active substances from implantable and other devices are described in U.S. Pat. Nos. 6,096,070; 5,824,049; 5,624,411; 5,609,629; 5,569,463; 5,447,724; and 5,464,650. The use of stents for drug delivery within the vasculature is described in PCT Publication No. WO 01/01957 and U.S. Pat. Nos. 6,099,561; 6,071,305; 6,063,101; 5,997,468; 5,980,551; 5,980,566; 5,972,027; 5,968,092; 5,951,586; 5,893,840; 5,891,108; 5,851,231; 5,843,172; 5,837,008; 5,769,883; 5,735,811; 5,700,286; 5,679,400; 5,649,977; 5,637,113; 5,591,227; 5,551,954; 5,545,208; 5,500,013; 5,464,450; 5,419,760; 5,411,550; 5,342,348; 5,286,254; and 5,163,952. Biodegradable materials are described in U.S. Pat. Nos. 6,051,276; 5,879,808; 5,876,452; 5,656,297; 5,543,158; 5,484,584; 5,176,907; 4,894,231; 4,897,268; 4,883,666; 4,832,686; and 3,976,071. The use of hydrocylosiloxane as a rate limiting barrier is described in U.S. Pat. No. 5,463,010. Methods for coating of stents are described in U.S. Pat. No. 5,356,433. Coatings to enhance biocompatibility of implantable devices are described in U.S. Pat. Nos. 5,463,010; 5,112,457; and 5,067,491. Energy based devices are described in U.S. Pat. Nos. 6,031,375; 5,928,145; 5,735,811; 5,728,062; 5,725,494; 5,409,000, 5,368,557; 5,000,185; and 4,936,281. Magnetic processes, some of which have been used in drug delivery systems, are described in U.S. Pat. Nos. 5,427,767; 5,225,282; 5,206,159; 5,069,216; 4,904,479; 4,871,716; 4,501,726; 4,357,259; 4,345,588; and 4,335,094.

The tube can be made from any material capable of expansion by a stent positioned on its interior and meeting the regulatory requirements for a material placed in a living body. The material is a material capable of releasing a drug or having a biological effect on surrounding or contacted tissue in the body. For example, the material can induce endothelization in a damaged arterial wall, or can elute a drug that inhibits restenosis or hyperplasia. Accordingly, the tube can be made from extracellular matrix material, or a synthetic extracellular matrix material. Other materials that can be used to make the tube include any biodegradable or bioerodable matrix materials employed for controlled release of drugs including, for example, poly-l-lactic acid/poly-e-caprolactone copolymer, polyanhydrides, polyorthoesters, polycaprolactone, poly vinyl acetate, polyhydroxybutyrate/polyhyroxyvalerate copolymer, polyglycolic acid, polyactic/polyglycolic acid copolymers and other aliphatic polyesters, among a wide variety of polymeric substrates available for devices that can be place in a human body.

The tube may also be fashioned of a combination of materials, for example including an extracellular matrix component along with other synthetic polymeric materials. The extracellular matrix material can be derived from a mammal, and intact, or can be an emulsion of extracellular matrix material that is mixed or extruded with or placed or wrapped around an extrudable polymer shaped in a tube. Native extracellular matrix scaffolds, and the proteins that form them, are found in their natural environment, the extracellular matrices of mammals. These materials are prepared for use in mammals in tissue grafts procedures. Small intestine submucosa (SIS) is described in U.S. Pat. No. 5,275,826, urinary bladder submucosa (UBS) is described in U.S. Pat. No. 5,554,389, stomach submucosa (SS) is described in U.S. Pat. No. 6,099,567, and liver submucosa (LS) or liver basement membrane (LBM) is described in U.S. Pat. No. 6,379,710, to name some of the extracellular matrix scaffolds presently available for explanting procedures. In addition, collagen from mammalian sources can be retrieved from matrix containing tissues and used to form a matrix composition. Extracellular matrices can be synthesized from cell cultures as in the product manufactured by Matrigel™. In addition, dermal extracellular matrix material, subcutaneous extracellular matrix material, large intestine extracellular matrix material, placental extracellular matrix material, ornamentum extracellular matrix material, heart extracellular matrix material, and lung extracellular matrix material, may be used, derived and preserved similarly as described herein for the SIS, SS, LBM, and UBM materials. Other organ tissue sources of basement membrane for use in accordance with this invention include spleen, lymph nodes, salivary glands, prostate, pancreas and other secreting glands. In general, any tissue of a mammal that has an extracellular matrix can be used for developing an extracellular matrix component of the invention.

When using collagen-based synthetic extracellular matrix materials, the collagenous matrix can be selected from a variety of commercially available collagen matrices or can be prepared from a wide variety of natural sources of collagen. Collagenous matrix for use in accordance with the present invention comprises highly conserved collagens, glycoproteins, proteoglycans, and glycosaminoglycans in their natural configuration and natural concentration. Collagens can be from animal sources, from plant sources, or from synthetic sources, all of which are available and standard in the art.

Native extracellular matrices are prepared with care that their bioactivity for myocardial tissue regeneration is preserved to the greatest extent possible. Key functions that may need to be preserved include control or initiation of cell adhesion, cell migration, cell differentiation, cell proliferation, cell death (apoptosis), stimulation of angiogenesis, proteolytic activity, enzymatic activity, cell motility, protein and cell modulation, activation of transcriptional events, provision for translation events, inhibition of some bioactivities, for example inhibition of coagulation, stem cell attraction, and chemotaxis. Assays for determining these activities are standard in the art. For example, material analysis can be used to identify the molecules present in the material composition. Also, in vitro cell adhesion tests can be conducted to make sure that the fabric or composition is capable of cell adhesion.

The matrices are generally decellularized in order to render them non-immunogenic. A critical aspect of the decellularization process is that the process be completed with some of the key protein function retained, either by replacement of proteins incidentally extracted with the cells, or by adding exogenous cells to the matrix composition after cell extraction, which cells produce or carry proteins needed for the function of tissue regeneration in vivo.

Synthetic extracellular matrices can be formed using synthetic molecules that polymerize much like native collagen and which form a scaffold environment that mimics the native environment of mammalian extracellular matrix scaffolds. According, such materials as polyethylene terephthalate fiber (Dacron), polytetrafluoroethylene (PTFE), glutaraldehyde-cross linked pericardium, polylactate (PLA), polyglycol (PGA), hyaluronic acid, polyethylene glycol (PEG), polyethelene, nitinol, and collagen from non-animal sources (such as plants or synthetic collagens), can be used as components of a synthetic extracellular matrix scaffold. The synthetic materials listed are standard in the art, and forming hydrogels and matrix-like materials with them is also standard. Their effectiveness can be tested in vivo as sited earlier, by testing in mammals, along with components that typically constitute native extracellular matrices, particularly the growth factors and cells responsive to them.

The extracellular matrix-like materials are described generally in the review article “From Cell-ECM Interactions to Tissue Engineering” Rosso et al, Journal of Cellular Physiology 199:174-180 (2004). In addition, some extracellular matrix-like materials are listed here. Particularly useful biodegradable and/or bioabsorbable polymers include polylactides, poly-glycolides, polycarprolactone, polydioxane and their random and block copolymers. Examples of specific polymers include poly D,L-lactide, polylactide-co-glycolide (85:15) and polylactide-co-glycolide (75:25). Preferably, the biodegradable and/or bioabsorbable polymers used in the fibrous matrix of the present invention will have a molecular weight in the range of about 1,000 to about 8,000,000 g/mole, more preferably about 4,000 to about 250,000 g/mole. The biodegradable and/or bioabsorbable fiberizable material is preferably a biodegradable and bioabsorbable polymer. Examples of suitable polymers can be found in Bezwada, Rao S. et al. (1997) Poly(p-Dioxanone) and its copolymers, in Handbook of Biodegradable Polymers, A. J. Domb, J. Kost and D. M. Wiseman, editors, Hardwood Academic Publishers, The Netherlands, pp. 29-61. The biodegradable and/or bioabsorbable polymer can contain a monomer selected from the group consisting of a glycolide, lactide, dioxanone, caprolactone, trimethylene carbonate, ethylene glycol and lysine. The material can be a random copolymer, block copolymer or blend of monomers, homopolymers, copolymers, and/or heteropolymers that contain these monomers. The biodegradable and/or bioabsorbable polymers can contain bioabsorbable and biodegradable linear aliphatic polyesters such as polyglycolide (PGA) and its random copolymer poly(glycolide-co-lactide-) (PGA-co-PLA). The FDA has approved these polymers for use in surgical applications, including medical sutures. An advantage of these synthetic absorbable materials is their degradability by simple hydrolysis of the ester backbone in aqueous environments, such as body fluids. The degradation products are ultimately metabolized to carbon dioxide and water or can be excreted via the kidney. These polymers are very different from cellulose based materials, which cannot be absorbed by the body.

Other examples of suitable biocompatible polymers are polyhydroxyalkyl methacrylates including ethylmethacrylate, and hydrogels such as polyvinylpyrrolidone, polyacrylamides, etc. Other suitable bioabsorbable materials are biopolymers which include collagen, gelatin, alginic acid, chitin, chitosan, fibrin, hyaluronic acid, dextran, polyamino acids, polylysine and copolymers of these materials. Any glycosaminoglycan (GAG) type polymer can be used. GAGs can include, e.g., heparin, chondroitin sulfate A or B, and hyaluronic acid, or their synthetic analogues. Any combination, copolymer, polymer or blend thereof of the above examples is contemplated for use according to the present invention. Such bioabsorbable materials may be prepared by known methods.

The tube may be a single layer of material, or may be multiple layers of the same or different material. The latter configuration may be beneficial in circumstances where a finely controlled timing of drug release is desired. For example, an outer layer of the tube (that contacts the lumen wall) can comprise an agent that prepares the lumen surface for healing processes. A next layer having a different drug can contact the lumen wall once the first layer has dissolved or biodegraded or otherwise exhausted its drug eluting potential, and so on for multiple layers. A layer on the interior of the tube, for example, can comprise and release a drug to facilitate non-thrombotic passage of the blood through the lumen.

The stent can be any self-expanding or expandable stent and can be made of metal or metal alloy or any material commonly used to make stents. Stent construction and design is well known in the art. See above for examples of stents known in the art. Stent materials can be any material appropriate for the construction and use of a stent, including, for example metal and metal alloy, for example nitinol. Generally, elasticity and/or expandability of the stent that is constructed are a key feature of the stent. Once in place the stent needs to be able to hold its structure so that the lumen can heal adjacent to it.

The procedure for placing the tube in the lumen of a living body includes several steps, the most basic of which is introducing the catheter into the lumen and detaching the tube from the catheter. These goals can be accomplished by any means feasible. Typically, a standard guidewire is advanced into the lumen across the lesion of interest with sufficient room to place a stent. A delivery catheter having the tube (still attached, but detachable) is advanced over the guidewire to place the tube portion at the lesion. A stent catheter carrying a stent (the stent can be, e.g., either alone and self expanding or disposed over a balloon) is then back-loaded over the guidewire but disposed within the delivery catheter and advanced to the lesion inside the tube. The stent is expanded, e.g. either by inflation of a balloon, or by a self-expanding means intrinsic to or within the stent, e.g. a spring-like capability in the stent, and so contacts the interior wall of the tube. As the stent continues to expand, the detachable tube expands with the stent, and then becomes trapped or sandwiched between the stent outer diameter and the inner diameter or surface of the lumen. During the expansion sequence, the perforations or attachments around the circumference of the tube will tear and yield therefore providing for the detachment of the tube from the catheter.

After confirming detachment of the expanded tube, the stent balloon is deflated and withdrawn from the catheter shaft. If another mechanism other than an expanding balloon is used, then that expanding and delivery mechanism is likewise withdrawn. After the stent catheter is removed, the detachable tube delivery catheter is removed. Correct sizing of the tube length and stent length is taken into consideration in order to match the length of the lesion or blocked area in the lumen. The stent diameter size is also important so that it can contact and exert pressure on the tube, and force the tube expansion and maintain that expansion to the point of contact of the lumen wall.

A primary advantage of a tube disposed in contact with a stent is that the tube can be used with any commonly manufactured stent. Additionally, the usually rigorous processing of a drug eluting stent is obviated because there is not coating required for the stent and thus the present invention can employ less costly bare metal stents in lieu of drug eluting stents. The detachable tube will perform the function of delivering drug to the site of defect in the lumen while the stent that expands within it and holds it in place against the lumen wall will provide support architecture at the site of defect. If the detachable tube is made of extracellular matrix material, the therapeutic nature of the extracellular matrix material as it remodels into adjacent healthy parent tissue may restore the lumen to an original healthy state, while the remaining stent will maintain a support architecture for the healing tissue.

Construction of the devices of the invention is accomplished by standard catheter construction with regard to the tube delivery catheter and the stent delivery catheter. For example, the luer and tube delivery catheter shaft are constructed using conventional techniques typically used in the manufacture of catheter products. The catheter shaft can be a single lumen extruded polymer affixed with a conventional luer. The inner diameter of the catheter shaft should be capable of receiving and allowing for free movement of a commercialized stent/balloon catheter along its entire length. The detachable tube can be fixed to the catheter shaft using conventional techniques like adhesives, heat shrink tubing, sewing, overmolding and the like. The detachable tube can be attached to the inner or outer diameter spaced at intervals sufficient to allow for the detachment of the tube by expansion of the stent via balloon inflation or by the spring force of a self-expanding stent. Another means of detachment of the tube is allowing for the detachment by way of radial force imparted on the detachable tube sufficient enough to overcome the fixing means. For example, the detachment could be accomplished by overcoming the adhesive forces of the fixing adhesive, detachment by radial expansion greater than the radial force imparted by the heat shrink tubing, and by tearing or yielding of the detachable tube material or threads used to affix the detachable tip to the catheter shaft.

There are many ways to construct the detachable tube. The detachable tube can be formed with a sheet of material, for example extracellular matrix or other therapeutic material, then rolled into a tube where the two opposing or overlapping edges can be sewn together using conventional practices. The detachable tube can be extruded as a tube wherein the therapeutic material can be forced through an opening provided by the extruding internal shape (for example a rod or mandrel) and the extruding external shape (for example a ring or dye head). The detachable tube can be shaped for example by dipping, spraying or electrostatic processes wherein the material is a fluid, gel, powder, or emulsification capable of adhering to a mold shape. The detachable tube would be formed around the mold shape and after processing could then be removed from the mold shape as a tubular component.

The tube can biodegrade slowly over time in the body after placement therein. Where the tube comprises extracellular matrix, the matrix material can promote healing and generation of healthy tissue at the site of defect. The tube can comprise other biodegradable materials and may also comprise drug containing or drug eluting materials. The drugs that may be placed or incorporated within the tube materials include any drug believed to be efficacious in treatment of the lumen defect, including any drug having an in vivo release profile compatible with the goals of the treatment. Thus, common drugs that may be deployed in this luminal environment include drugs to promote endothelization of the lumen wall, and anti-thrombotic drugs to prevent blockage by drug clot formation in the lumen and elsewhere in the body. Anti-proliferative drugs may also be used to prevent restenosis in vascular lumens. Other drugs appropriate for the particular treatment objectives may also be used. The tube material or materials (e.g. where the tube is layered using more than one material or is itself a combination of materials) may also present more than one drug, e.g. where the drugs can work in concert, or where each administered drug is directed to a different but compatible therapeutic objective at the site of defect or in the body generally. A tube comprised of more than one layer of material can present a different drug to the body in each layer.

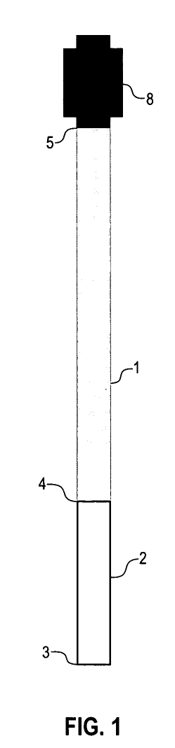

Turning now to the Figures, FIG. 1 depicts the basic catheter 1 having a distal detachable portion or tube 2 detachable at perforations or loose attachments 4. The distal end of the tube 3 is unattached to the catheter in this embodiment. A self-expanding stent or a stent coated balloon can be placed into tube 2 through the catheter shaft 1 and expanded within tube 2 to break perforations 4 and release the distal detachable tube 2 from the catheter 1.

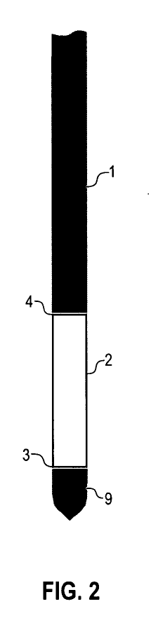

FIG. 2 depicts a dilator embodiment of the expandable tube system. Catheter 1 is positioned to deliver tube 2 that is detachable from the catheter at point 4, and open-ended at point 3. Tube 2 can have perforations or other detachment means at position 4 so that upon expansion of tube 2, the tube detaches from catheter 1 by breaking the perforations or loose attachments at position 4. The dilator 9 is disposed within the catheter shaft 1, and also within tube 2 and provides a means for an atraumatic introduction of the device into a body lumen. With dilator 9, there is no need for a guidewire to guide the catheter positioning. A stent containing catheter (not shown) can be introduced into catheter 1 and passed over the dilator at a position interior to tube 2.

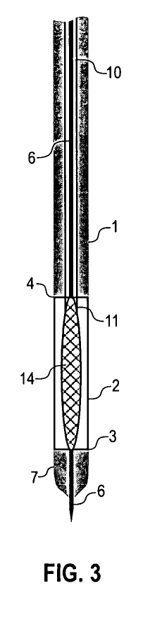

FIG. 3 depicts a nose cone embodiment wherein catheter 1 has a detachable portion or tube 2 located approximately at the distal end of the catheter 1. The catheter 1 has a nose cone portion 7 that is disposed distally to the tube 2 for the purpose of atraumatic introduction of the device into a body lumen. Guidewire 6 is positioned within the nose cone 7 and catheter 1 for introducing the catheter 1, and attached tube 2. Subsequently, a stent catheter (not shown) can be positioned over the guidewire 6 for the introduction of the stent within the tube 2. Perforations or attachments 4 and 3 provide a means for separation of the tube 2 from the catheter 1 by placing a self-expanding stent or a balloon expanding stent to break the perforations or attachments at 4 and 3, thus releasing the tube 2 into the lumen and with full expansion of the stent (not shown), tube 2 is pressed against the lumen wall and held there by the interior pressure established with the stent.

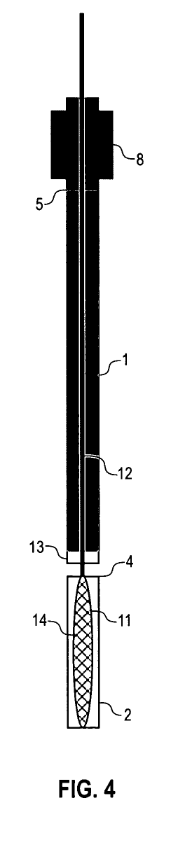

FIG. 4 depicts the catheter apparatus 1 having an expanding stent 11 and stent catheter 12 disposed within it. Stent 11 is placed within tube 2 and depicted in the figure is the partial inflation of a balloon 14 having stent 11 covering it. Controls for operations in the catheter can be conducted from luer 8 that attaches to catheter 1 at position 5. Stent catheter 12 can be introduced over a guidewire (not shown) or dilator (also not shown) in order to place the stent 11 and balloon 14 within tube 2. Tube 2 detaches at point 4 from catheter 1 upon expansion of stent 11 using balloon 14 within tube 2. Tube 2 detaches at point 4 from catheter 1 upon expansion of stent 11 using balloon 14 within tube 2 that causes pressure and the tube is released by breaking perforations or attachments at 4 that connect to the catheter at position 13.

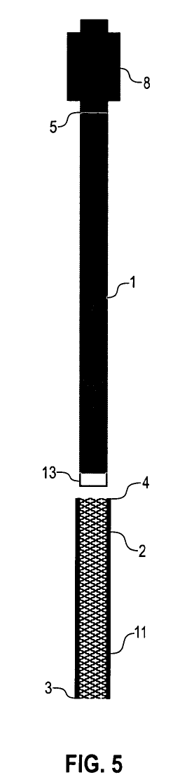

FIG. 5 depicts an expanded stent 11 contacting the interior walls of tube 2 and detached from catheter 1 at position 4 where perforations or other attachments have broken from catheter attachment region 5. Luer 8 is disposed at the proximal end of catheter 1, connecting to the catheter at position 5. Fully expanded stent 11 contacts the interior walls of tube 2 and the pressure from stent 11 places tube 2 in full contact with the walls of the body lumen.

The invention provides a method of repairing a defect in a lumen surface by

placing an expandable tube having an interior wall and an exterior wall at a site of defect in a body lumen. Disposed within the tube is an expandable stent. Generally the tube is placed in the lumen using a delivery catheter (for example placed at the site by backloading the delivery catheter over a guidewire that is already in place in the lumen) and an expandable stent is placed within the tube using a stent catheter that is similarly backloaded onto the guidewire and advanced until the stent is disposed within the tube at the site of defect. The stent is then expanded from within the tube either by inflation of a balloon or by activating a spring mechanism that allows the stent to exert force on the tube and cause it to expand outwardly. A fmal position for the system is having the exterior wall of the tube contacting the lumen surface, and having the stent pushed up against and contacting the interior wall of the tube. After such placement and positioning is achieved, the stent catheter, tube delivery catheter and guidewire can be removed.

The lumen having the defect and treatable using a device or method of the present invention can be any body lumen, for example a vascular lumen, e.g. an artery or a vein. Other body lumens include the colon and small intestine, and various lumens of or connecting to organs in the body. The defect in the lumen can be for example a lesion or blockage, or lumen wall otherwise in disrepair or having a disease component. In general any lumen capable of receiving and holding a stent and having a defect capable of treatment using a device or method of the invention can be a lumen treated using the present devices and methods.

The invention contemplates also a method of making a device for delivery to a body lumen, wherein the device comprises a tube comprising an expandable therapeutic polymeric material, the tube having an interior wall and an exterior wall, and proximal and distal ends. The tube is formed from the expandable polymeric material and perforations or detachment points are engineered into the points of contact of the tube with the portion of the material that remains part of the catheter. The tube with its detachment points is then placed at a distal end of the catheter, and delivered as described above. Forming the tube can comprise for exampled, extruding, sewing, laminating, pressing, freeze-drying, gluing, or molding. Single or a combination of materials can be used to form the tube, including, e.g. extracellular matrix derived from a mammal, synthetic extracellular matrix, an extruded material, a biodegradable material, and a drug eluting material.

Placement of the tube comprising therapeutic materials, including e.g. drug eluting materials in a lumen of a body accomplishes a method of drug delivery. The tube that is placed in the body comprises a therapeutic polymeric material having at least one bioactive agent or drug capable of release in the lumen of the body. Thus, by engineering a drug or more than one drug into the materials of the tube, a delivery of drug to the body is effected when the tube is placed in the lumen and the material of the tube is allowed to biodegrade and bioabsorb along with release of the drug carried within the material.

Although the foregoing invention has been described in detail for purposes of clarity of understanding, it will be obvious that certain modifications may be practiced within the scope of the appended claims. The references sited herein are incorporated by reference in their entirety.

What is claimed is:

1. A device comprising a tube comprising extracellular matrix, the tube having an interior wall, an exterior wall, a distal end, and a proximal end, wherein the tube is detachable from a catheter at the proximal end, and can be placed in a body lumen.

2. The device of claim 1, wherein the tube comprises perforations at the proximal end for detachment from the catheter.

3. The device of claim 2, wherein the tube is detachable from the catheter by breaking the perforations.

4. The device of claim 2, wherein the tube also comprises perforations at the distal end for detachment from the catheter.

5. The device of claim 4, wherein the tube is detachable from the catheter by breaking the perforations.

6. The device of claim 1, wherein the tube is expandable.

7. The device of claim 1, wherein the tube is expandable upon application of pressure on the interior wall of the tube.

8. The device of claim 2, wherein the tube is expandable upon application of pressure on the interior wall and the expansion breaks the perforations so that the tube detaches from the catheter.

9. The device of claim 6, wherein the application of pressure on the interior wall comprises expansion of an expandable stent disposed within the tube, said stent contacting the interior wall.

10. The device of claim 9, wherein the expandable stent disposed within tube can expand until the exterior wall of the tube contacts a surface of the body lumen.

11. The device of claim 10, wherein the stent is self-expanding.

12. The device of claim 10, wherein the stent is expandable with a balloon.

13. The device of claim 1, wherein the extracellular matrix is derived from a mammal.

14. The device of claim 1, wherein the extracellular matrix is synthetic.

15. The device of claim 1, wherein the extracellular matrix is a combination of synthetic and mammalian extracellular matrices.

16. The device of claim 1, wherein the material further comprises a material selected from the group consisting of an extruded material, a biodegradable material, and a drug eluting material.

17. A catheter device having a distal end comprising a detachable portion, the detachable portion comprising a tube placeable in the lumen of a body, the tube having an interior wall and exterior wall, and a distal end and a proximal end detachable from the catheter, the tube comprising an expandable therapeutic polymeric material.

18. The catheter device of claim 17, wherein the detachable portion is located at or near the distal end of the catheter.

19. The catheter device of claim 17, wherein the material is selected from the group consisting of extracellular matrix derived from a mammal, synthetic extracellular matrix, an extruded material, a biodegradable material, and a drug eluting material.

20. The catheter device of claim 17, wherein the tube comprises perforations at the proximal end of the tube for detachment from the catheter.

21. The catheter device of claim 17, wherein the tube comprises perforations at the distal and proximal ends of the tube for detachment from the catheter.

22. The catheter device of claim 17, wherein the tube is detachable from the catheter by interior expansion of the tube that breaks the perforations and detaches the tube from the catheter.

23. The catheter device of claim 17, further comprising an expandable stent disposed within the tube.

24. The catheter device of claim 17, wherein the stent is self-expanding or expandable with a balloon.

25. A method of repairing a defect in a lumen surface comprising:
a. placing an expandable tube having an interior wall and an exterior wall and an expandable stent disposed within the tube at the site of defect, and
b. expanding the expandable stent until the exterior wall of the tube is in contact with the lumen surface and the stent is in contact with the interior wall of the tube.

26. The method of claim 25, wherein the tube comprises a therapeutic polymeric material.

27. The method of claim 26, wherein the material is selected from the group consisting of extracellular matrix derived from a mammal, synthetic extracellular matrix, an extruded material, a biodegradable material, and a drug eluting material.

28. The method of claim 25, wherein the stent comprises a metal or metal alloy.

29. The method of claim 25, wherein the stent is expandable with an inflatable balloon.

30. The method of claim 25, wherein the stent is self-expanding.

31. A method of repairing a defect in a lumen surface comprising:
a. placing a guidewire in the lumen proximal to the defect,
b. sliding over the guidewire a first catheter comprising an expandable tube, the tube comprising perforations for detachment from the catheter as an intact tube, the tube having an interior wall and an exterior wall,
c. inserting an expandable stent within the expandable tube using a second catheter disposed within the first catheter, the second catheter also sliding over the guidewire, and
d. expanding the stent within the tube until the exterior wall of the tube is in contact with the lumen surface and the stent is in contact with the interior wall of the tube.

32. The method of claim 31, wherein during the expanding step, the stent expands within the tube and the tube detaches from the first catheter.

33. The method of claim 31, wherein the tube comprises a therapeutic polymeric material.

34. The method of claim 31, wherein the material is selected from the group consisting of extracellular matrix derived from a mammal, synthetic extracellular matrix, an extruded material, a biodegradable material, and a drug eluting material.

35. The method of claim 31, wherein the stent is self expanding.

36. The method of claim 31, wherein the stent is expanded by an expansion means.

37. The method of claim 36, wherein the stent expansion means comprises a balloon.

38. The method of claim 31, wherein the stent comprises a metal or metal alloy.

39. The method of claim 38, wherein the stent comprises nitinol.

40. A method of making a device for delivery to a body lumen, the device comprising a tube comprising an expandable therapeutic polymeric material, the tube having an interior wall and an exterior wall, and proximal and distal ends, comprising:
a) forming a tube of the expandable therapeutic polymeric material,
b) applying perforations in the tube at the proximal end to make the tube detachable from a delivery catheter, and
c) attaching the perforated tube to a catheter for delivery to the body lumen.

41. The method of claim 40, further comprising applying perforations in the tube at the distal end.

42. The method of claim 40, wherein forming comprises a process selected from the group consisting of extruding, sewing, laminating, pressing, freeze-drying, gluing, and molding.

43. The method of claim 40, wherein the expandable therapeutic polymeric material is selected from the group consisting of extracellular matrix derived from a mammal, synthetic extracellular matrix, an extruded material, a biodegradable material, and a drug eluting material.

44. A method of drug delivery comprising placing a tube in the lumen of a living body, wherein the tube comprises a therapeutic polymeric material having at least one bioactive agent or drug capable of release in the lumen of the body.

45. The method of claim 44, wherein a stent is expanded within the tube to place the tube in contact with a lumen surface.
